(19) United States
(12) Patent Application Publication
(10) Pub. No.: US 2011/0015129 A1
(43) Pub. Date: Jan. 20, 2011

(54) NEW PAEDIATRIC INDICATIONS FOR DIRECT THROMBIN INHIBITORS

(75) Inventors: Andreas CLEMENS, Wiesbaden (DE); Lisa CRONIN, Oakville (CA)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/891,184

(22) Filed: Sep. 27, 2010

Related U.S. Application Data
(63) Continuation of application No. 11/779,032, filed on Jul. 17, 2007, now abandoned.

(30) Foreign Application Priority Data
Jul. 17, 2006 (EP) 06117347
Feb. 15, 2007 (EP) 07102514

(51) Int. Cl. A61K 38/58; A61K 31/4439; A61K 31/397; A61K 31/4709; A61P 9/00; A61P 9/10; A61P 9/06; A61P 3/10; A61P 35/00; A61P 7/02; A61P 7/00; A61P 3/00; A61P 25/28; A61P 13/12; A61P 25/00; A61P 29/00

(52) U.S. Cl. 514/14.8; 514/338; 514/210.17; 514/314

(57) ABSTRACT

The invention relates to new paediatric indications for direct thrombin inhibitors such as dabigatran etexilate.

The present invention relates to novel indications for direct thrombin inhibitors (DTI), processes for preparing pharmaceutical compositions for treating said diseases and methods of treating them.

### DETAILED DESCRIPTION OF THE INVENTION

Direct thrombin inhibitors according to the invention include
(1) 1-methyl-2-(4-amidinophenylaminomethyl)-benzimidazol-5-yl-carboxylic acid-(N-2-pyridyl-N-2-hydroxycarbonylethyl)-amide known as dabigatran having the structure

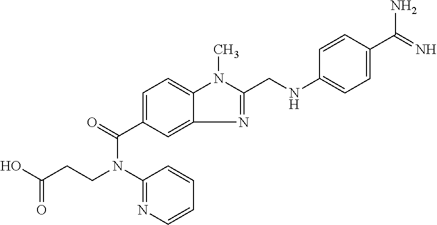

(2) ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate known as dabigatran etexilate having the following structure

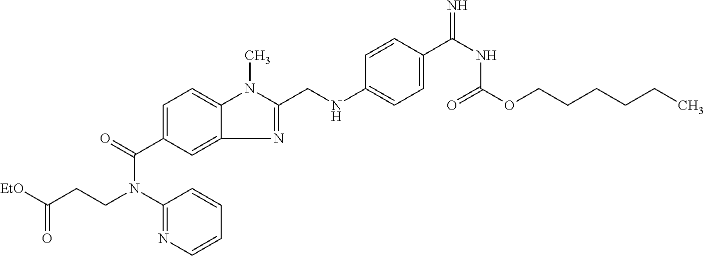

(3) 1-methyl-2-[4-(N-hydroxyamidino)-phenylaminomethyl]-benzimidazol-5-yl-carboxylic acid-(N-2-pyridyl-N-2-ethoxycarbonylethyl)-amide having the structure

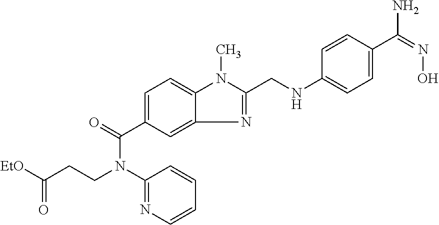

(4) melagatran (inogatran),
(5) ximelagatran,
(6) hirudin,
(7) hirolog and
(8) argatroban,
   optionally in the form of tautomers, racemates, enantiomers, diastereomers, pharmacologically acceptable acid addition salts, solvates, hydrates or prodrugs thereof.

Preferred direct thrombin inhibitors are dabigatran, dabigatran etexilate and 1-methyl-2-[4-(N-hydroxyamidino)-phenylaminomethyl]-benzimidazol-5-yl-carboxylic acid-(N-2-pyridyl-N-2-ethoxycarbonylethyl)-amide, and the tautomers, racemates, enantiomers, diastereomers, pharmacologically acceptable acid addition salts, solvates, hydrates and prodrugs thereof.

More preferred are dabigatran and dabigatran etexilate, and the tautomers, racemates, enantiomers, diastereomers, pharmacologically acceptable acid addition salts, solvates, hydrates and prodrugs thereof.

Most preferred is dabigatran etexilate, and the tautomers, racemates, enantiomers, diastereomers, pharmacologically acceptable acid addition salts, solvates, hydrates and prodrugs thereof, particularly its acid addition salt with methanesulfonic acid.

All active components should be used in effective amounts.

The active compounds (1) to (3) are disclosed in the prior art, e.g. in WO 98/37075 and WO 04/014894. The acid addition salt of dabigatran etexilate with methanesulfonic acid is described in WO 03/074056. Additional salts of dabigatran etexilate are mentioned in the experimental part. Specific polymorphs and a hemihydrate of acid addition salt of dabigatran etexilate with methanesulfonic acid is described in WO 2005/028468. Examples for pharmaceutical composition containing dabigatran etexilate are disclosed in WO 03/074056, WO 2005/018615 and WO 2005/023249.

Prodrugs of the drugs mentioned above are such derivatives containing one or more groups capable of being cleaved in vivo, particularly a group which can be converted in-vivo into a carboxy group or/and a group capable of being cleaved in vivo from an imino or amino group. Compounds containing two groups capable of being cleaved in vivo are so-called double prodrugs. Groups which can be converted in-vivo into a carboxy group and groups capable of being cleaved in vivo from an imino or amino group are disclosed e.g. in WO 98/37075, being herewith incorporated by reference, as well as in other WO publications cited hereinbefore in connection with specific antithrombotics.

It is understood that the direct thrombin inhibitor according to the invention may be used in a form selected from tautomers, optical isomers, enantiomers, racemates, diastereomers, pharmacologically acceptable acid addition salts, solvates or hydrates, as far as such forms exist, depending on the individual compound. If multiple enantiomers exist, the use in form of a substantially pure enantiomer is preferred.

Pharmacological acceptable acid addition salts of the direct thrombin inhibitors listed above comprise salts selected from the group consisting of the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrolactate, hydrooxalate, hydrosuccinate, hydrobenzoate and hydro-p-toluolsulphonate, preferably hydrochloride, hydrobromide, hydrosulphate, hydrophosphate, hydromaleate, hydrofumarate and hydromethansulphonate. Some of the direct thrombin inhibitors may add more than one equivalent acid, e.g. two equivalents. The salts of hydrochloric acid, methanesulfonic acid, maleic acid, benzoic acid and acetic acid are especially preferred.

A preferred embodiment are the salts of dabigatran etexilate with hydrochloric acid, maleic acid, tartaric acid, salicylic acid, citric acid, methanesulfonic acid and malonic acid, the enantiomers, mixtures and hydrates thereof. Particularly preferred are tartaric acid, salicylic acid, methanesulfonic acid and citric acid as well as the enantiomers, mixtures and hydrates thereof. The most preferred salt of is the methanesulfonic acid addition salt of dabigatran etexilate.

The following terms are used synonymously:

salt with hydrochloric acid—hydrochloride
salt with maleic acid—maleate
salt with tartaric acid—tartrate
salt with salicylic acid—salicylate
salt with citric acid—citrate
salt with malonic acid—malonate
salt with methanesulfonic acid—methanesulfonate

Any reference to a direct thrombin inhibitor within the scope of the present invention should be understood as a reference to any specific direct thrombin inhibitor selected from compounds (1) to (8) mentioned hereinbefore.

A preferred embodiment of the invention relates to new indications of the active substance ethyl 3-[(2-{[4-(hexyloxycarbonylamino-imino-methyl)-phenylamino]-methyl}-1-methyl-1H-benzimidazole-5-carbonylypyridin-2-yl-amino]-propionate, the salts, the enantiomers, the mixtures and the hydrates thereof. This active substance with the chemical formula

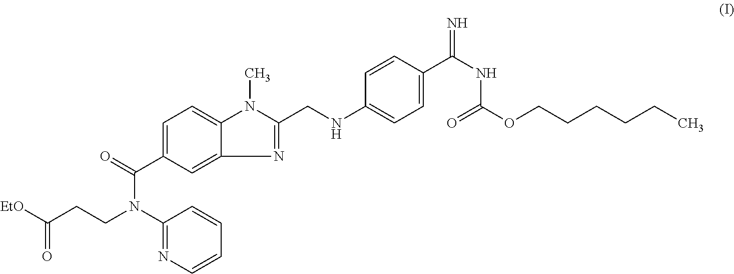

is already known from WO 98/37075, wherein compounds with a thrombin-inhibiting and thrombin time-prolonging activity are disclosed, under the name 1-methyl-2-[N-[4-(N-n-hexyloxycarbonylamidino)phenyl]-amino-methyl]-benzimidazol-5-yl-carboxylic acid-N-(2-pyridyl)-N-(2-ethoxycarbonylethyl)-amide. The compound of formula I is a double prodrug of the compound

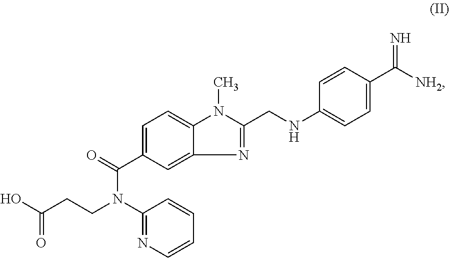

i.e. the compound of formula I is first converted into the actual effective compound, namely the compound of formula II, in the body. The main type of indication for the compound of chemical formula I is the post-operative prophylaxis of deep vein thrombosis and the prevention of strokes.

Surprisingly, the direct thrombin inhibitors like e.g. dabigatran etexilate cannot only be used effectively for the post-operative prophylaxis of deep vein thrombosis and the prevention of strokes, but are also suitable for the prevention and/or treatment of children.

In particular the invention relates to the use of a compound, optionally in the form of tautomers, racemates, enantiomers, diastereomers, pharmacologically acceptable acid addition salts, solvates, hydrates or prodrugs thereof, selected from the group consisting of dabigatran, dabigatran etexilate, 1-methyl-2-[4-(N-hydroxyamidino)-phenylaminomethyl]-benzimidazol-5-yl-carboxylic acid-(N-2-pyridyl-N-2-ethoxycarbonylethyl)-amide, melagatran (inogatran), ximelagatran, hirudin, hirolog and argatroban for preparing a medicament for the treatment and/or prophylaxis of a disease selected from among thrombosis and/or venous thromboembolic events (VTE) in children, preferably VTE selected from among
primary VTE prevention,
secondary VTE prevention and
VTE treatment in children.

In another embodiment the invention relates to the use of the compounds mentioned hereinbefore for preparing a medicament for the treatment and/or prophylaxis of stroke in children,
preferably for the treatment of non haemorhagic stroke in children or for stroke prevention in children selected from among
   primary and secondary stroke prevention in children with atrial fibrillation and
   primary and secondary stroke prevention in children at elevated risk for stroke (children after transitoric ischemic attack (TIA) or stroke and post myocard infarction or acute coronary syndrome in children, children with very low ejection fraction of the heart).

In yet another embodiment the invention relates to the use of the compounds mentioned hereinbefore for preparing a medicament for the treatment and/or prophylaxis of myocardial infarction (sometimes also named acute coronary syndrome [ACS]) in children,
preferably ACS resp. myocardial infarction in children
   with/after stent implantation,
   with percutaneous coronary intervention (PCI) without stent implantation
   and without PCI in children.

The treatment and/or prophylaxis of myocardial infarction resp. ACS may either begin immediately after the event (acute treatment) or a certain time after the event (e.g. after myocardial infarction, post-MI) (chronic therapy, secondary prevention).

In yet another embodiment the invention relates to the use of the compounds mentioned hereinbefore for preparing a medicament for the treatment and/or prophylaxis of myocardial infarction in children, in particular myocardial infarction in children with arterio coronary venous bypass (ACVB) and also in children after thrombolysis.

In another embodiment the invention relates to the use of the compounds mentioned hereinbefore for preparing a medicament for the treatment and/or prophylaxis of thrombosis or thromboembolic events in children with an off pump coronary artery by pass grafting surgery.

In another embodiment the invention relates to the use of the compounds mentioned hereinbefore for preparing a medicament for the treatment and/or prophylaxis of graft thrombosis in children, in particular graft thrombosis in ACVB children and also in children after thrombolysis.

In another embodiment the invention relates to the use of the compounds mentioned hereinbefore for preparing a medicament for the treatment and/or prophylaxis of stroke in children, particularly for the prevention of stroke in children with atrial fibrillation.

In another embodiment the invention relates to the use of the compounds mentioned hereinbefore for preparing a medicament for the treatment and/or prophylaxis of post-operative prophylaxis of deep vein thrombosis (DVT) in children.

In another embodiment the invention relates to the use of the compounds mentioned hereinbefore for preparing a medicament for the treatment and/or prophylaxis of thrombosis or thromboembolic events in children, in particular in off pump coronary artery bypass and/or grafting surgery.

In another embodiment the invention relates to the use of the compounds mentioned hereinbefore for preparing a medicament for the treatment and/or prophylaxis of stent thrombosis in children, in particular stent thrombosis in PCI patients and also in patients after thrombolysis

In another embodiment the invention relates to the use of the compounds mentioned hereinbefore for preparing a medicament for the treatment and/or prophylaxis of elevated cardiovascular risk in children, preferably elevated cardiovascular risk in children under treatment with antihypertensive and/or lipid lowering drugs, in children with elevated inflammatory status, in children with elevated coagulant parameters (e.g. PAI 1) or in children with diabetes mellitus.

In another embodiment the invention relates to the use of the compounds mentioned hereinbefore for preparing a medicament for the treatment and/or prophylaxis of congenital heart disease in children, in particular open foramen ovale, congenital heart failure, congenital disposition of the vessels and vessel anormalities (e.g. aortic isthmus stenosis) in children.

In another embodiment the invention relates to the use of the compounds mentioned hereinbefore for preparing a medicament for the treatment and/or prophylaxis of diseases selected from among disorders in children, e.g. due to artificial heart valves, arrhythmia, heart failure, hypertrophic obstuctive cardiomyopathy (HOCM) and diabetes mellitus.

In another embodiment the invention relates to the use of the compounds mentioned hereinbefore for preparing a medicament for the treatment and/or prophylaxis of peripheral arterial disease (PAD) in children, in particular of peripheral arterial disease
in children suffering from diabetes mellitus,
in children with or without implanted stent(-s) in the peripheral vessel(-s) and in children who underwent peripheral bypass surgery.

In another embodiment the invention relates to the use of the compounds mentioned hereinbefore for preparing a medicament for the treatment and/or prophylaxis of a disease selected from among brain micro vessel disease and pulmonary infarction in children.

In another embodiment the invention relates to the use of the compounds mentioned hereinbefore for preparing a medicament for the prevention and/or treatment of shunt thrombosis, catheter thrombosis (including central venous line [CVL]) and thromboembolic events in children, in particular in children on dialysis with shunt or without shunt and in the dialysis machine.

In another embodiment the invention relates to the use of the compounds mentioned hereinbefore for the treatment and/or prophylaxis of pulmonary embolism (PE) in children, in particular of PE in children with higher risk for PE (e.g. congenital coagulopathy, children after multiple pulmonary embolisms) and in children with deep venous thromboembolism (DVT) and/or any other kind of VTE.

In another embodiment the invention relates to the use of the compounds mentioned hereinbefore for preparing a medicament for the treatment and/or prophylaxis of thrombosis, venous thromboembolic events (VTE), pulmonary embolism (PE) and deep venous thromboembolism (DVT) (anticoagulant therapy) in medical care children (immobilized children), in particular
in children immobilized after any kind of surgery,
in children immobilized after any kind of accident or trauma,
in immobilized children with additional risk factors for VTE,
in children with cancer, particularly in children with acute lymphoblastic leukaemia (ALL),
in children with heart failure,
in children with multiple sclerosis (MS) or
in children with another diagnosis which results in immobilization of the child.

In another embodiment the invention relates to the use of the compounds mentioned hereinbefore for preparing a medicament for the treatment and/or prophylaxis of the diseases mentioned in this application occurring in pregnant girls, in particular stroke, heart failure (high risk gravidas), congenital hypercoagulation disease and haemolysis in pregnant girls, as well as for the treatment and/or prophylaxis of elevated liver enzymes and low platelets (HELLP) syndrome (in pregnant girls).

In another embodiment the invention relates to the use of the compounds mentioned hereinbefore for preparing a medicament for the treatment and/or prophylaxis of acute or chronic arterial thromboembolism (for example due to cardiac catheterisation, central venous line (CVL) etc.) in children.

Herein, prophylaxis includes application prior to surgery resp. catherisation as well as during the surgery resp. catherisation.

In another embodiment the invention relates to the use of the compounds mentioned hereinbefore for preparing a medicament for the treatment and/or prophylaxis of congenital heart disease in children, in particular postoperative congentital heart disease in children and VTE in children.

In another embodiment the invention relates to the use of the compounds mentioned hereinbefore for preparing a medicament for the treatment and/or prophylaxis of venous thromboembolism and/or VTE in children with cancer (e.g. acute lymphoblastic leukaemia (ALL)), particularly in children under chemotherapy involving Asparaginase.

In another embodiment the invention relates to the use of the compounds mentioned hereinbefore for preparing a medicament for the treatment and/or prophylaxis of a disease selected from the group consisting of:
neurodegenerative disease,
brain micro vessel disease,
diseases which are mediated via PAR 1 to PAR 4 receptors
and oxidative stress induced by thrombin in children.

In another preferred embodiment the invention is related to the use of the compounds mentioned hereinbefore for preparing a medicament for the treatment and/or prophylaxis of
Haematological diseases,
heparin induced thrombocythompenia (HIT),
disseminated intravascular coagulation (DIC) in children.

In another preferred embodiment the invention is related to the use of the compounds mentioned hereinbefore for preparing a medicament for the treatment and/or prophylaxis of a disease selected from among
thrombosis in children,
thrombosis and/or venous thromboemobilc events in polychemotherapy (particularly in polychemotherapy involving Asparaginase) in children suffering from cancer, particularly in children suffering form leukaemia such as acute lymphoblastic leukaemia (ALL).

In another preferred embodiment the invention is related to the use of the compounds mentioned hereinbefore for preparing a medicament for the treatment and/or prophylaxis of central vein thrombosis (CVT) in children.

In another preferred embodiment the invention is related to the use of the compounds mentioned hereinbefore for preparing a medicament for the treatment and/or prophylaxis of HIV encephalitis in children suffering from human immunodefience virus (HIV).

In another preferred embodiment the invention is related to the use of the compounds mentioned hereinbefore for preparing a medicament for the treatment and/or prophylaxis of rheumatoid disorders in children, in particular
rheumatoid arthritis and
systemic lupus erythematodes (SLE) in children.

In another preferred embodiment the invention is related to the use of the compounds mentioned hereinbefore for preparing a medicament for the treatment and/or prophylaxis of Tinnitus Aurium in children.

In another preferred embodiment the invention is related to the use of the compounds mentioned hereinbefore for preparing a medicament for the treatment and/or prophylaxis of kidney disease in children, in particular
proteinuria (urinary albumin excretion) in patients with chronic kidney disease and
proteinuria (urinary albumin excretion) in patients with Diabetes and albuminuria.

The thrombin inhibitors listed above are useful for the prevention and/or treatment of events provoked by the above-mentioned diseases (like VTE, PE), optimize the blood flow to organs or regions, and/or are suitable for direct treatment of the diseases.

A preferred embodiment is the use of the direct thrombin inhibitors according to the invention for the preparation of a medicament for treating or preventing VTE associated with any one of the diseases mentioned above resp. below.

The term “patient” as used in this application is to be understood as referring to children. Within the meaning of the instant invention children are patients with an age below 18 years, preferably, below 16 years, more preferably below 14 years, yet more preferably below 12 years. In particular children may be patients with an age in the range of 1 to 10 years.

A preferred group of patients are children up to 5 years old; another preferred group of patients are children between 6 and 10 years; yet another preferred group of patients are children between 11 and 16 years.

Preferred indications are:
treatment of non-haemorhagic stroke in children,
primary and secondary stroke prevention in children with very low ejection fraction of the heart;
acute stroke in children,
treatment and/or prophylaxis of myocardial infarction resp. acute coronary syndrome (ACS) in children, preferably ACS resp. myocardial infarction in children
   with/after stent implantation,
   with percutaneous coronary intervention (PCI) without stent implantation,
   without PCI in children;
treatment and/or prophylaxis of thrombosis, venous thromboembolic events (VTE), pulmonary embolism (PE) and deep venous thromboembolism (DVT) in medical care children (immobilized children), in particular
   in children immobilized after any kind of surgery,
   in children immobilized after any kind of accident or trauma,
   in children with additional risk factors for VTE,
   in children with cancer,
   in children with heart failure,
   in children with multiple sclerosis (MS) or
   in children with another diagnosis which results in immobilization of the child;
treatment and/or prophylaxis of elevated cardiovascular risk in
   children, preferably elevated cardiovascular risk in children under treatment with antihypertensive and/or lipid lowering drugs,
   children with elevated inflammatory status,
   children with elevated coagulant parameters (e.g. PAI 1) or in
   children with diabetes mellitus;
treatment and/or prophylaxis of congenital heart disease in children, in particular
   open foramen ovale,
   congenital heart failure,
   congenital disposition of the vessels and
   vessel anormalities in children;
treatment and/or prophylaxis of cardiovascular disorders in children due to
   artificial heart valves in children,
   arrhythmia in children,
   heart failure in children,
   hypertrophic obstuctive cardiomyopathy (HOCM) in children or
   diabetes mellitus in children;
treatment and/or prophylaxis of peripheral arterial disease (PAD) in children, in particular PAD
   in children with diabetes mellitus,
   in children with or without implanted stent(-s) in the peripheral vessel(-s) and
   in children who underwent peripheral bypass surgery;
treatment and/or prophylaxis of brain micro vessel disease in children;
treatment and/or prophylaxis of pulmonary infarction in children;
treatment and/or prophylaxis of shunt thrombosis in children, particularly in children on dialysis,
treatment and/or prophylaxis of catheter thrombosis in children, particularly in children on dialysis,
treatment and/or prophylaxis of thromboembolic events in the dialysis machine;
treatment and/or prophylaxis of pulmonary embolism (PE) in children, in particular of PE in children with higher risk for PE (e.g. congenital coagulopathy, children after multiple pulmonary embolisms); treatment and/or prophylaxis of stroke in pregnant girls, of heart failure in pregnant girls (high risk gravidas), of congenital hypercoagulation disease in pregnant girls, of haemolysis in pregnant girls and of elevated liver enzymes and low platelets (HELLP) syndrome in pregnant girls;
treatment and/or prophylaxis of thrombosis or thromboembolic events in children with an off pump coronary artery bypass grafting;
1) CNS-field
   a. neurodegenerative disease (e.g. Alzheimer disease) in children
   b. brain micro vessel disease in children
   c. diseases which are mediated via PAR 1 to PAR 4 receptors in children
   d. oxidative stress induced by thrombin in children
2) Haematologic disease
   a. Heparin induced thrombocythompenia in children
   b. children with elevated coagulant parameters (e.g. PAI 1)
   c. Disseminated intravascular coagulation (DIC) in children
3) Cancer
   a. Primary and secondary prevention and/or treatment of cancer in children
   b. Prevention of thrombosis in polychemotherapy in children, particularly in polychemotherapy including Asparaginase,
   c. Prevention of thrombosis in children
   d. Treatment of thrombosis in children
   e. Mortality reduction as mono-therapy and in combination with anti-cancer agents in children
4) Ophtamology
   a. Central vein thrombosis (CVT) in children
5) Human Immunodefience Virus (HIV) patients
   a. HIV encephalitis in children
6) Rheumatoid disorders in children
   a. Rheumatoid arthritis in children
   b. Systemic Lupus erythematodes (SLE) in children
7) Children with transplantation
8) Children with implants
   a. shunt prosthesis in children on dialysis
   b. prosthesis of the vessel (Aorta etc.) in children
9) Children with Tinnitus Aurium
10) Children with kidney disease
   a. Proteinuria (urinary albumin excretion) in patients with chronic kidney disease
   b. Proteinuria (urinary albumin excretion) in patients with Diabetes and albuminuria.

In another embodiment the invention relates to the use of the compounds mentioned hereinbefore for preparing a medicament for the treatment and/or prophylaxis in children of one or several of the diseases mentioned hereinbefore, wherein the disease is associated with VTE.

The direct thrombin inhibitor, optionally used in form of its pharmaceutically acceptable acid addition salts, may be incorporated into the conventional pharmaceutical preparation in solid, liquid or spray form. The composition may, for example, be presented in a form suitable for oral, topical, lingual, rectal, parenteral administration or for nasal inhalation: preferred forms includes for example, capsules, tablets, coated tablets, ampoules, suppositories and nasal spray.

The active ingredient may be incorporated in excipients or carriers conventionally used in pharmaceutical compositions such as, for example, talc, arabic gum, lactose, gelatine, magnesium stearate, corn starch, acqueous or non acqueous vehicles, polyvinyl pyrrolidone, semisynthetic glicerides of fatty acids, benzalconium chloride, sodium phosphate, EDTA, polysorbate 80. The compositions are advantageously formulated in dosage units, each dosage unit being adapted to supply a single dose of the active ingredient. The dosis range applicable per day is between 0.1 mg to 600 mg, preferably between 50 mg to 300 mg/day. Each dosage unit may conveniently contain from 0.1 mg to 200 mg, preferably from 50 mg to 150 mg.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g of. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g of. with the addition of preservatives such as p-hydroxybenzoates, or stabilisers such as alkali metal salts of ethylenediamine tetraacetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

### EXAMPLES

The Examples which follow illustrate the present invention without restricting its scope:

The starting material dabigatran etexilate (ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate) may for example be prepared as described in International Application WO 98/37075, Example 113.

### Example 1

### Hydrochloride of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate

125 mg (1.59 mmol) of acetyl chloride were added to 5 ml ethanol with stirring. The solution thus obtained was then added dropwise at ambient temperature to a solution of 1.0 g (1.59 mmol) of ethyl 3-[(2-{[4-(amino-hexyloxycarbonyliminomethyl)-phenylamino]-methyl}-1-methyl-1H-benzimidazole-5-carbonyl)-pyridin-2-yl-amino]-propionate and stirred for a further two hours. The mixture was then evaporated down completely, the residue was first of all triturated after the addition of approx. 5 ml ethyl acetate and suction filtered, then stirred overnight in approx. 10 ml acetone, suction filtered, washed with a little acetone and diethyl ether and then dried at 60° C. in vacuo.

Yield: 86% of theory

Melting point: 135° C.

### Example 2

### Citric acid salt of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate

210 mg (1.0 mmol) of citric acid hydrate, dissolved in 10 ml ethyl acetate, were added dropwise at ambient temperature with stirring to a solution of 628 mg (1.0 mmol) of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate in 45 ml ethyl acetate. A yellow precipitate formed. The mixture was stirred overnight, the product was then suction filtered, washed with a little ethyl acetate and diethyl ether and dried at approx. 50° C. in vacuo.

Yield: 83% of theory

Melting point: approx. 170° C. (with decomposition)

### Example 3

### Tartaric acid salt of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate

150 mg (1.0 mmol) of L(+)-tartaric acid, dissolved in 5 ml absolute ethanol, were added dropwise at ambient temperature with stirring to a solution of 628 mg (1.0 mmol) of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate in 50 ml ethyl acetate. A fine precipitate was formed. The suspension was stirred for a further two hours, then the product was suction filtered, washed with a little cold ethyl acetate and diethyl ether and dried in vacuo at approx. 50° C.

Yield: 72% of theory

Melting point: approx. 160° C. (with decomposition)

### Example 4

### Malonic acid salt of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate

104 mg (1.0 mmol) of malonic acid, dissolved in 10 ml ethyl acetate, were added dropwise at ambient temperature, with stirring, to a solution of 628 mg (1.0 mmol) of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate in 50 ml ethyl acetate. After approx. one hour a fine precipitate formed. The suspension was stirred for a further three hours, the product was then suction filtered, washed with a little cold ethyl acetate and diethyl ether and dried in vacuo at approx. 50° C.

Yield: 79% of theory

Melting point: 100° C.

### Example 5

### Maleic acid salt of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate

116 mg (1.0 mmol) of maleic acid, dissolved in 10 ml ethyl acetate, were added dropwise, with stirring, at ambient temperature, to a solution of 628 mg (1.0 mmol) of ethyl 3-[(2-{[4-(amino-hexyloxycarbonylimino-methyl)-phenylamino]-methyl}-1-methyl-1H-benzimidazole-5-carbonyl)-pyridin-2-yl-amino]-propionate in 50 ml ethyl acetate. A precipitate formed. The suspension was stirred for a further three hours, then the product was suction filtered, washed with a little cold ethyl acetate and diethyl ether and dried in vacuo at approx. 50° C.

Yield: 93% of theory

Melting point: 120° C.

### Example 6

### Ethyl-3-[(2-{[4-(hexyloxycarbonylamino-imino-methyl)-phenylamino]-methyl}-1-methyl-1H-benzimidazole-5-carbonyl)-pyridin-2-yl-amino]-propionate salicylate

A solution of 1.38 g (10.0 mmol) of salicylic acid in 20 ml acetone was added dropwise with stirring at 35-40° C. to a solution of 6.28 g (10.0 mmol) of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base (prepared as described in WO 98/37075), in 45 ml acetone. After a few minutes the product began to crystallise out and it was diluted with 65 ml acetone. Within 30 minutes the mixture was cooled to ambient temperature, then the precipitate was suction filtered, washed with approx. 40 ml acetone and dried at 40° C. in the circulating air dryer.

Yield: 94% of theory

Melting point: 155° C.

### Example 7

### Dry Ampoule Containing 75 mg Active Substance Per 10 ml

### Composition:

| | |
|---|---|
| active substance | 75.0 mg |
| mannitol | 50.0 mg |
| water for injections | ad 10.0 ml |

### Preparation:

Active substance and mannitol are dissolved in water. After packaging the solution is freeze-dried. To produce the solution ready for use for injections, the product is dissolved in water.

### Example 8

### Dry Ampoule Containing 35 mg of Active Substance Per 2 ml

### Composition:

| | |
|---|---|
| Active substance | 35.0 mg |
| Mannitol | 100.0 mg |
| water for injections | ad 2.0 ml |

### Preparation:

Active substance and mannitol are dissolved in water. After packaging, the solution is freeze-dried.

To produce the solution ready for use for injections, the product is dissolved in water.

### Example 9

### Tablet Containing 50 mg of Active Substance

### Composition:

| | |
|---|---|
| (1) Active substance | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

### Preparation:

(1), (2) and (3) are mixed together and granulated with an aqueous solution of (4). (5) is added to the dried granulated material. From this mixture tablets are pressed, biplanar, faceted on both sides and with a dividing notch on one side. Diameter of the tablets: 9 mm.

### Example 10

### Tablet Containing 350 mg of Active Substance

### Composition:

| | |
|---|---|
| (1) Active substance | 350.0 mg |
| (2) Lactose | 136.0 mg |
| (3) Maize starch | 80.0 mg |
| (4) Polyvinylpyrrolidone | 30.0 mg |
| (5) Magnesium stearate | 4.0 mg |
| | 600.0 mg |

### Preparation:

(1), (2) and (3) are mixed together and granulated with an aqueous solution of (4). (5) is added to the dried granulated material. From this mixture tablets are pressed, biplanar, faceted on both sides and with a dividing notch on one side. Diameter of the tablets: 12 mm.

### Example 11

### Capsules Containing 50 mg of Active Substance

### Composition:

| | |
|---|---|
| (1) Active substance | 50.0 mg |
| (2) Dried maize starch | 58.0 mg |
| (3) Powdered lactose | 50.0 mg |
| (4) Magnesium stearate | 2.0 mg |
| | 160.0 mg |

### Preparation:

(1) is triturated with (3). This trituration is added to the mixture of (2) and (4) with vigorous mixing.

This powder mixture is packed into size 3 hard gelatine capsules in a capsule filling machine.

### Example 12

### Capsules Containing 350 mg of Active Substance

### Composition:

| | |
|---|---|
| (1) Active substance | 350.0 mg |
| (2) Dried maize starch | 46.0 mg |
| (3) Powdered lactose | 30.0 mg |
| (4) Magnesium stearate | 4.0 mg |
| | 430.0 mg |

### Preparation:

(1) is triturated with (3). This trituration is added to the mixture of (2) and (4) with vigorous mixing.

This powder mixture is packed into size 0 hard gelatine capsules in a capsule filling machine.

### Example 13

### Suppositories Containing 100 mg of Active Substance

1 suppository contains:

| | |
|---|---|
| Active substance | 100.0 mg |
| Polyethyleneglycol (M.W. 1500) | 600.0 mg |
| Polyethyleneglycol (M.W. 6000) | 460.0 mg |
| Polyethylenesorbitan monostearate | 840.0 mg |
| | 2,000.0 mg |

### Example 14

| | Percentage composition | | | | | |
|---|---|---|---|---|---|---|
| | | | Active | | per | per |
| | Core | Separating | substance | | capsule | capsule |
| | material | layer | layer | Total | [mg] | [mg] |
| Tartaric acid | 61.3 | — | — | 61.3 | 176.7 | 353.4 |
| Gum arabic | 3.1 | 2.8 | | 5.9 | 17.0 | 34.0 |
| Talc | — | 5.6 | 3.2 | 8.8 | 25.4 | 50.7 |
| Hydroxyhydroxypropyl- | — | — | 4.0 | 4.0 | 11.5 | 23.1 |
| cellulose | | | | | | |
| Active substance (based | — | — | 20.0 | 20.0 | 50.0 | 100.0 |
| on the base) | | | | | | |
| Total | | | | 100.0 | 288.3 | 576.5 |

### Example 15

| | Percentage composition | | | | | |
|---|---|---|---|---|---|---|
| | | | Active | | per | per |
| | Core | Separating | substance | | capsule | capsule |
| | material | layer | layer | Total | [mg] | [mg] |
| Tartaric acid | 38.5 | — | — | 38.5 | 55.5 | 166.5 |
| Gum arabic | 1.9 | 1.7 | | 3.6 | 5.2 | 15.6 |
| Talc | — | 3.5 | 6.4 | 9.9 | 14.3 | 42.8 |
| Hydroxyhydroxypropyl- | — | — | 8.0 | 8.0 | 11.5 | 34.6 |
| cellulose | | | | | | |
| Active substance (based | — | — | 40.0 | 40.0 | 50.0 | 150.0 |
| on the base) | | | | | | |
| Total | | | | 100.0 | 144.2 | 432.5 |

The preparation and the structure of the pellets according to Examples 14 and 15 is described in detail in WO 03/074056.

We claim:

1. A method for the treatment and/or prophylaxis in children of a disease selected from the group consisting of:
non-haemorhagic stroke;
primary and secondary stroke prevention in children with very low ejection fraction of the heart;
acute stroke;
acute coronary syndrome (ACS);
myocardial infarction;
elevated cardiovascular risk;
congenital heart disease;
artificial heart valves;
arrhythmia;
heart failure;
hypertrophic obstuctive cardiomyopathy (HOCM);
diabetes mellitus;
peripheral arterial disease (PAD);
brain micro vessel disease;
pulmonary infarction;
shunt thrombosis;
catheter thrombosis;
thromboembolic events in the dialysis machine;
off pump coronary artery bypass grafting;
pulmonary embolism (PE);
medical care children (immobilized children);
cancer;
stroke in pregnant girls,
heart failure in pregnant girls (high risk gravidas);
congenital hypercoagulation disease in pregnant girls;
hemolysis, elevated liver enzymes and low platelets (HELLP) syndrome in pregnant girls;
neurodegenerative disease;
brain micro vessel disease;
diseases which are mediated via PAR 1 to PAR 4 receptors;
oxidative stress induced by thrombin;
haematology;
heparin induced thrombocythompenia;
thrombosis in poly chemotherapy;
central vein thrombosis (CVT);
HIV encephalitis;
rheumatoid disorders;
Tinnitus Aurium and
kidney disease,
comprising the step of administering to a child in need thereof a therapeutically effective amount of a compound, optionally in the form of tauto-mers, racemates, enantiomers, diastereomers, pharmacologically acceptable acid addition salts, solvates, hydrates or prodrugs thereof, selected from the group consisting of dabigatran, dabigatran etexilate, 1-methyl-2-[4-(N-hydroxyamidino)-phenylaminomethyl]-benzimidazol-5-yl-carboxylic acid-(N-2-pyridyl-N-2-ethoxycarbonylethyl)-amide, melagatran (inogatran), ximelagatran, hirudin, hirolog and argatroban.

2. The method according to claim 1, wherein the disease is associated with VTE.

3. The method according to claim 1 or 2, wherein the compound is selected from the group consisting of dabigatran, dabigatran etexilate and 1-methyl-2-[4-(N-hydroxyamidino)-phenylaminomethyl]-benzimidazol-5-yl-carboxylic acid-(N-2-pyridyl-N-2-ethoxycarbonylethyl)-amide.

4. The method according to one of claims 1-3, wherein the compound is selected from the group consisting of dabigatran and dabigatran etexilate or a pharmacologically acceptable acid addition salt thereof.

5. The method according to one of claims 1-4, wherein the compound is dabigatran etexilate or a pharmacologically acceptable acid addition salt thereof.

6. The method according to one of claims 1-5, wherein the compound is the acid addition salt of dabigatran etexilate with methanesulfonic acid.

7. The method according to one of claims 1-6, wherein the compound is applied in a dose range between 0.1 mg to 600 mg per day.
